(19) United States
(12) Patent Application Publication
(10) Pub. No.: US 2010/0330568 A1
(43) Pub. Date: Dec. 30, 2010

(54) ASSAY AND METHOD FOR THE ASSESSMENT OF RESPONDERS AND NON-RESPONDERS TO NK CELL MODULATION BY IMMUNOGLOBULIN THERAPY

(76) Inventors: Stefan Meuer, Heidelberg (DE); Thomas Giese, Heidelberg (DE); Christian Jacobi, Heidelberg (DE); Jürgen Römisch, Gramatneusiedl (AT); Stefan Haag, Jona (CH)

Correspondence Address: JACOBSON HOLMAN PLLC, 400 SEVENTH STREET N.W., SUITE 600, WASHINGTON, DC, 20004, US

(21) Appl. No.: 12/735,313

(22) PCT Filed: Jan. 9, 2009
(86) PCT No.: PCT/EP2009/050216

§ 371 Date: Aug. 26, 2010

(30) Foreign Application Priority Data
Jan. 9, 2008 (EP) 08100251.1

(51) Int. Cl. C12Q 1/68; G01N 33/68; C12Q 1/02

(52) U.S. Cl. 435/6; 435/7.24; 435/29

(57) ABSTRACT

A method of determining a patient's susceptibility for NK cell modulation by immunoglobulins in response to a treatment of a disease or prophylaxis of a disease with immunoglobulins wherein a modulation of natural killer cells caused by said immunoglobulins is determined.

The invention pertains to a method of determining a patient's susceptibility to immunoglobulins in response to a treatment of a disease or prophylaxis of a disease.

Natural killer cells (NK cells) are part of the innate immune and host defence system. A possible important function of NK cells is the attack of intruders like viruses and tumor cells. The mechanism is assumed to be based on the recognition in particular of those cells bearing low or no levels of the major histocompatibility complex MHC class I, which is usually involved in the presentation of specific portions of the intruder after cell internalisation and proteolysis. By such characteristic NK cells may differentiate between infected from non-infected cells to target their attack. Distinct viruses even prevent the export of MHC class I molecules evading the specific host defence but thereby becoming a target for NK cells attack.

Specific HLA alleles expressed by human cells indicate self and in combination with the aforementioned characteristic of recognition an “own” body cell is protected providing an additional safety mechanism from self-attack.

A very effective mechanism of killing is mediated through direct NK target cell contact. The NK cell releases molecules towards the foreign cells. These molecules are released from NK cell granulae, therefore this effect is called degranulation, liberating stored effector molecules, NK cells use the so called ‘Perforin’, which forms a channel in the target cell membrane or coat leading to severe damage of the target all even to its ‘death’. Other molecules are involved in this attack, supporting the destroying effect by other mechanisms like proteolysis, in which granzyme B is involved also being released by NK cell degranulation. Release of those signalling compounds, including cytokines, attrack and activate other cells resulting in a composite attack to intruders and finally down-modulating the defence reaction.

This killing power can be utilised for distinct cancer treatment approaches when the tumour cells are attacked and destroyed by NK cells. Therefore, distinct compounds and interventions have been searched to support NK cell activity and to direct' them more specifically against the tumour cells.

NK cells are an important part of the host defence. However, when dysregulation of these cells occurs, e.g. as reason or in the course of different diseases, they can direct themselves against “self-structures” with significant pathophysiological consequences, like attack of organ structures and oligodendrocytes in the periphery and brain. Although the mechanisms are not fully understood leading to such mis-direction and self-attack, regaining control of these cells and reducing their killing power to a physiologically reasonable level is required. As killing efficiency is tightly connected to degranulation and release of effector molecules, it is an aim to control such degranulation to reduce damage. It is therefore the objective to identify and use compounds which can modulate NK cell actions reducing/preventing said actions and subsequent damage.

Immunoglobulins are a natural part of the immune system, which recognise targets comprising many different compounds, structures and those also as part of cellular structures, to recognise and often neutralise their biological effect. The immune complexes are cleared fast leading to the elimination of a ‘foreign’ molecule, Accordingly, recognition, binding and removal function is doubtless of essential importance, which is substantiated by the fact that patients lacking (or have reduced) immunoglobulin levels are prone to serious and recurrent infections. Beyond this protection function towards intruders, immunoglobulins bear important regulatory function in balancing and regulating the immune system. Immunoglobulin products, usually derived from pooled blood or plasma donations and prepared according processes well known to the expert, are used for the treatment of IMID (immune mediated inflammatory diseases) and so called AID (autoimmune diseases), while those definitions may express identical or overlapping features of a disease. The immunoglobulin concentrates are usually applied intravenously (IVIG) or subcutaneously (SCIG), but may be also intra-muscularly, inhaled, intra-ocularly, orally or topically.

The effectivity of intravenous immunoglobulin (IVIG) in autoimmune diseases was first described in the 1950s, when it was used to treat patients suffering from idiopathic thrombocytopenic purpura. In the meantime many clinical studies demonstrated a beneficial effect of IVIG in autoimmune diseases. Among others, IVIG therapy is proven in Guillain-Barré syndrome (GBS), Kawasaki syndrome chronic inflammatory demyelinising polyneuropathy, myasthenia gravis and corticosteroid-resistant dermatomyositis (Ephrem et al., 2005; Kazatchkine and Kaveri, 2001; Boros et al., 2005). A positive effect of IVIG on disease progression relapse rate and MRI enhancing lesions in multiple sclerosis (MS) was demonstrated by clinical studies as well (Sorensen P S, 2003; Sorensen et al., 2002).

The complete mechanism of action of IVIG is still unclear but seems to invoke the modulation of expression and function of F_{c} receptors, interference with complement activation, modulation of T- and B-cell-activation, -differentiator and -effector functions (Ephrem et al., 2005; Kazatchkine and Kaveri, 2001; Boros et al., 2005).

While IVIG prophylaxis and treatment is successfully used in many patients, among them are cases who poorly or even not respond to IVIG application. At present: there are neither reliable test systems explaining such non-responsiveness nor facilitating a prediction whether immunoglobulin application will result in the expected efficiency in the patient. Recent studies showed that for instance in recurrent spontaneous abort, NK cell number and activity may predict abortion or normal pregnancy, i.e. high number/activity bearing a poor prognosis. Moreover a correlation was found with the effect of immunoglobulin tested in vitro addressing the NK cell killing activity level, However, the mechanism how NK cell activity is influenced by immunoglobulins in a direct or indirect manner remained unknown, thus the test brought forward was focused on the NK killing activity in the presence of a Cr⁵¹-labeled cell-line and corresponding Cr⁵¹ release, facilitating an estimation according to this parameter. Moreover it is speculative whether the above mentioned indication is representative for e.g. IMID (immune mediated inflammatory disease) indications.

Asphalter et al in Clin Exp Immunol 2000, 121, 506-514 discloses effects of in vivo IVIG replacement therapy and high-dose IVIG (2 g/kg body weight) on NK subsets. Also disclosed is an intracellular NK IFN-gamma assay wherein intracellular IFN-gamma was measured in NK cells before and after IVIG therapy (200-400 mg/kg every 3 weeks). At least one measurement of both assays was performed after therapy, which does not provide the possibility of discriminating between responders and none-responders.

Ruiz. et al. in Journal of Reproductive Immunology, 31 (1996), 125-141 pertains to the immunological mechanism of IVIG to inhibit NK activity in vitro when it was added to NK cytotoxicity assays using peripheral blood lymphocytes as targets. Reduced ⁵¹Cr-release indicated a reduction in NK cell toxicity. As Cr was bound unspecific to the cell surface it is doubtful that the measured radioactivity results from a degranulation.

Tha-In Thanyalak et al. in Blood, Vol 110, No 9, 9. November 2007 relates to dendritic cells (DC's) matured for 18 hours in the presence of IVIG and their influence on natural killer (NK) cells. The effect of these cells on NK cell phenotype was examined by determination of the expression of Fc-gamma-RIII after 5 days. INF-gamma production and degranulation of NK cells was also determined with cocultured IVIG-DC's after 48 hours and showed high contents of interferon-gamma. Most interesting is the observation that control samples of NK cells treated with IVIG without DC's showed only marginal activation. Thus, THA-IN concluded that only IVIG-DC's activate NK cell degranulation properly. Besides of the long-lasting assays (48 hours and 5 days) performed with different cells compared to the present application (IVIG-DC's are not used by the present invention) no indication can be found to use the findings for the determination of a persons susceptibility to immunoglobulins in response to a treatment of a disease. Additionally, the only comparable assay (NK cells treated with IVIG) showed contradictory results compared to the present application, unfortunately, no specific conditions were published, which makes a substantial comparison impossible (page 3257, left column, lines 3-7).

Kwak Joanne Y. H. et al. in EARLY PREGNANCY, Vol IV, pp 154-164 investigated the clinical effect of IVIG treatment in recurrent aborters with elevated NK cell levels while still receiving additional other treatments. NK cytotoxicity and expression of CD16+ was found to be significantly suppressed 7 days after IVIG infusion treatment. There was no indication found to use the findings for the determination of a person's susceptibility to immunoglobulins in response to a treatment of a disease.

EP-A-1801234 relates to diagnostic methods to predict whether a subject is predisposed for acquiring a disease or developing an autoimmune disease by use of recombinant nucleic acid constructs. Such constructs are not used by the present application.

Park-Min Kyung-Hyun et al. in IMMUNITY, vol. 26, no. 1, January 2007, pp 67-78 describes several investigations related to the influence of IVIG on cellular responses to interferon-gamma. These investigations were mainly based on observations of Wisteria monocytogenes and on macrophages, which are no NK cells.

### BRIEF DESCRIPTION OF THE INVENTION

A technical problem of the invention was to provide a method for differentiating responders from non-responders to an administration of immunoglobulin.

Surprisingly it was found that NK cells in the presence of immunoglobulins, exposed to in form of IVIG product are modulated in an in vitro whole blood test system. Among others the IFN-gamma release and NK cell degranulation are induced, while the killing activity is reduced. CD16 (FcγRIII) on the cell surface of NK cells is modulated as well.

The technical problem underlying the invention is solved by a method of determining a patient's susceptibility to immunoglobulins in response to a treatment of a disease or prophylaxis of a disease with immunoglobulins wherein a modulation of NK cells and an amount of a degranulation of natural killer cells caused by said immunoglobulins is determined.

The term degranulation is well known to the skilled person and has the following meaning: release of secretory granule contents by fusion with the plasma membrane.

Usually the presence of a NK cell target (like K562 labelled) cells is required to induce NK cell degranulation upon cell-cell contact, thereby affecting target cell killing by armament (see above) release from NK cell granulae. It was discovered that exposure of whole blood to immunoglobulins (IVIG) induces a degranulation of a comparable sensitive NK cell subset equivalent or more effective than upon cell-cell contact with target cells (as measured with K562). This leads to liberation of the granulae compounds into the extra cellular space. The extra cellular space can be simulated by a medium or a solution in the assay set-up. In particular, the amount of the degranulation of natural killer cells is determined in an in vitro method which can be established by the skilled person.

For example, whole blood was stimulated by IVIG-incubation or placebo control for a certain period of time such as about 3 hours. Then anti-human CD107a or isotype control was added and the sample was e.g. incubated with about 2×10⁵ K562 target cells at about 37° C. for about 3 hours. A control sample was incubated without target cells to detect spontaneous and IVIG induced degranulation. Thereafter samples were stained with anti-CD56 and anti-CD3 monoclonal antibodies, followed by a detection step, in particular by flow cytometric analysis.

The patient having a positive degranulation of NK cells is determined as susceptible to a NK cell modulation by treatment with immunoglobulins.

According to an embodiment of the invention determining a patient's susceptibility to immunoglobulins in response to a treatment of a disease or prophylaxis of a disease with immunoglobulins is performed by way of detection of a transcript level, a protein level or a transcript level and a protein level.

According to another embodiment of the invention the transcript level, the protein level or the transcript level and the protein level is quantitatively determined. For example the transcript level is determined by nucleic acid detection methods, such as polymerase chain reaction (PCR) and/or the protein level is determined using released natural killer cell proteins and/or proteins expressed on the natural killer cell surface.

In still another embodiment of the invention whole blood or blood cells are incubated in presence of a stimulant in presence or absence of immunoglobulins followed by measurement of event factors selected from the group showing NK cell modulation consisting of IFN-gamma, IP-10, F_{c}-gamma III receptor, granzyme B, perforin or combinations thereof for assessing individual responsiveness to the administration of immunoglobulin. The term “combinations” is fully understood by the skilled person as IFN-gamy and IP-10, IFN-gama and F_{c}-gamma III receptor, IFN-gamma and granzyme B, IFN-gamma and perforin; IP-10 and F_{c}-gamma III receptor, IP-10 and granzyme B, IP-10 and perforin; F_{c}-gamma III receptor and granzyme B, F_{c}-gamma III receptor and perforin; granzyme B and perforin; IFN-gama and IP-10 and F_{c}-gamma III receptor; IFN-gama, IP-10, granzyme B; IFN-gama and IP-10 and perforin; IFN-gamma and granzyme B and perforin; IP-10 and Fₑ-gamma III receptor and granzyme B; IP-10 and F_{c}-gamma III receptor and perforin; F_{c}-gamma III receptor and granzyme B and perforin; IFN-gama and IP-10 and F_{c}-gamma III receptor and granzyme B; IP-10 and F_{c}-gamma III receptor and granzyme B and perforin; IFN-gama and IP-10 and F_{c}-gamma III receptor and granzyme B and perforin.

The event factors which are measured are e.g. the abundance of the transcription and/or protein expression level, in particular quantitative measurements of the event factors.

In one embodiment of the invention the stimulant is selected from the group consisting of lipopolysaccharides (LPS), phorbol-12-myristate-13 acetate (PMA), ionomycin, monoclonal antibodies (mAbs) binding to cell surface proteins or combinations thereof. Although the term “combinations” is fully clear to the skilled person, the combinatory list is exemplified as: LPS and PMA, LPS and ionomycin; LPS and mAbs binding to cell surface proteins; PMA and ionomycin; PMA and mAbs binding to cell surface proteins; ionomycin and mAbs binding to cell surface proteins; LPS and PMA and ionomycin; LPS and ionomycin and mAbs binding to cell surface proteins; PMA and ionomycin and mAbs binding to cell surface proteins; LPS and PMA and ionomycin and mAbs binding to cell surface proteins.

Typically the amount of the immunoglobulins used in assays may vary from about 0.01 to about 100 mg/ml, in particular from about 1 to about 50 mg/ml.

The range from about 1 to about 50 mg/ml is well known to the skilled person who readily understands that at least each integer between the limits of 1 and 50 is encompassed by this terminology, in particular the skilled person reads in the range 1 to 50 the numerical combinations of 1, 2, 3, 4, 5, 6, 7, 8, 9, 10, 11, 12, 13, 14, 15, 16, 17, 18, 19, 20, 21, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50; or 2, 3, 4, 5, 6, 7, 8, 9, 10, 11, 12, 13, 14, 15, 16, 17, 18, 19, 20, 21, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 but also, for example, 1, 2, 3 or 2, 3, 5, 6 and any other combinations possible by just permutating the values lying in the range.

The results obtainable according to the method of the invention are relevant in the treatment of a disease selected from the group consisting of inflammatory mediated immune diseases, autoimmune diseases and allergies.

### DETAILED DESCRIPTION OF THE INVENTION

The degranulation reaction itself can be sensitively detected by

a.) utilizing the cellular re-structuring response (like surface molecule detection)
and/or
b.) the release of granulae containing compounds.

As an example for a.) CD107a (LAMP1) surface expression is known as a marker for NK cell degranulation. The increase of monitored signal (like fluorescence), measured by methods known to the experts, like specific detection of a labelled antibody, fragment or affinity ligand for flow cytometry (like FACS, fluorescent-activated cell sorting) indicates the degranulation efficacy upon IgG exposure, as described in more detail in example 1. Any other change of cellular marker associated with degranulation can be utilised to detect and quantify the degranulation efficacy and status induced by IgG exposure. Representatives of such NK cell granulae (lytic lysosomes) compounds are the proteins perforin and granzymes (the latter proteases, more specifically granzyme B), which can be quantified by for instance antigen detection systems like ELISA or direct enzymatic tests (for enzymes and proteases).

While the described reaction can be utilised to predict an IVIG mediated in vivo effect, it constitutes an important reaction involved in in vivo (physiological) regulation and an effect mediated by IVIG application. The induced “exhaustion” (release of NK cell armament) of poorly controlled NK cells directly by administered immunoglobulins can reduce the NK cell killing potency for a certain period of time and thereby diminish cell destructive potency against “self” structures in a dysregulated pathogenic situation. Consequently, pathophysiological events associated to or initiated by NK cell contribution are indicated for immunoglobulin prophylaxis and therapy by IVIG, SCIG and other forms of application known to the expert.

The identification of responders/non-responders of immunoglobulin prophylaxis and therapy can be achieved by other parameters, a combination of those and in combination with the degranulation assay. For this purpose a whole blood assay, but also on separated cells, can be utilised to predict immunoglobulin efficacy in vivo.

To those parameters belongs Interferon-gamma (IFN-gamma). Its mRNA (transcripts) is induced upon in vitro application of whole blood or e.g. NK cells, which also shows by an increase of protein levels produced by the cells, IP-10 (CXCL10, chemo attractant for T cells and NK cells among other functions) is usually under the control of IFN-gamma, i.e. stimulated by the latter when increased. Using lipopolysaccharide (bacteria derived) in a whole blood assay system and thereby mimicking a patho-physiological situation, both IFN-gamma and IP-10 are up-regulated on mRNA transcript (number) and protein (release) level. It was found that in the presence of added immunoglobulin (upon stimulation with LPS), the increase of IP-10 was reduced by immunoglobulins as compared to the control without immunoglobulins, whereas no relative reduction was observed for IFN-gamma. This effect is unexpected and is interpreted as part of the immune-modulating properties by immunoglobulins, thus leading to a partial uncoupling' of the IFN-gamma and IP-10 response, which can be interpreted as anti-inflammatory reaction. Correspondingly, this effect can be utilised in an ex vivo test system (whole blood from a potential IVIG recipient) to assess whether responsiveness to IVIG application is likely.

Furthermore, this parameter can be used in combination with assays on F_{c} gamma-receptor regulation, as immunoglobulins induce the down-modulation of F_{c}-gamma-R type III, the subset of which is reported to be associated with inflammatory reaction. Accordingly, reduction of transcripts and protein cell surface expression will direct physiology to the anti-inflammatory side.

Moreover, these parameters can be combined with assays on single nucleotid polymorphisms (SNP) like Interleukin-2 receptor (IL-2R), interleukin-7 receptor (IL7-R) and CD58 indicating the patients different genetic background.

The invention and respective assays are described in more detail by the following examples.

### Example 1

### NK Cell Degranulation Assay (CD107a)

### Method

100 μl human blood was diluted with 100 μl assay medium (IMDM with 10% FCS and penicillin/streptomycin) and incubated at least once with IVIG and for control at least once without IVIG for 3 h at 37° C. Then PE-Cy5 conjugated anti-CD107a mAb or isotype control was added and the sample was incubated with 2×10⁵ K562 target cells at 37° C. for 2-3 h. A control sample was incubated without target cells to detect spontaneous and IVIG induced degranulation. Thereafter samples were stained with FITC-conjugated anti-CD56 and PE-conjugated anti-CD3 mAbs, followed by flow cytometric analysis. Immunoglobulin (5% or 10% IVIG, Octagam, Octpharma ppGmbH, Vienna, Austria) was added to whole blood at 10 mg/ml. Maltose solution was used as a control of the IVIG containing maltose.

### Result

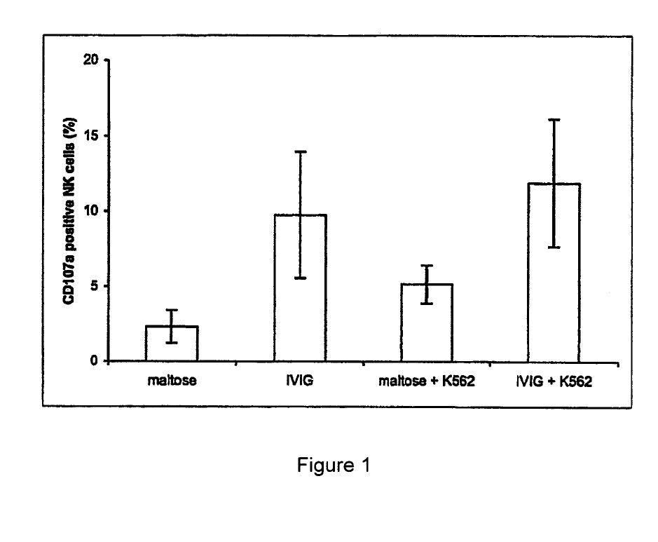

The induction of NK cell degranulation can be demonstrated by an increasing number of CD107a positive cells. The results for eight healthy subjects are shown in FIG. 1. In all healthy subjects a degranulation of NK cells was induced, which was found to be significantly more effective than the control (maltose). K562 cells were used for the assessment of NK killing activity (control: K562 cells+maltose). Notably, stronger effects were mediated by IVIG than by the standard K562 cells. A trend to slightly enhanced degranulation was observed using IVIG and K562 cells in combination.

### F_{c}-Gamma-III-Receptor (CD16) Expression on NK Cells

To measure F_{c}-gamma-III-receptor (CD16) on NK cells 100 μl of IVIg or control treated whole blood samples (prepared as described in example 1) were stained with PerCP-Cy5.5-conjugated anti-CD16, PE-Cy7-conjugated anti-CD56 and Pacific Blue-conjugated anti-CD3 followed by flow cytometric analysis.

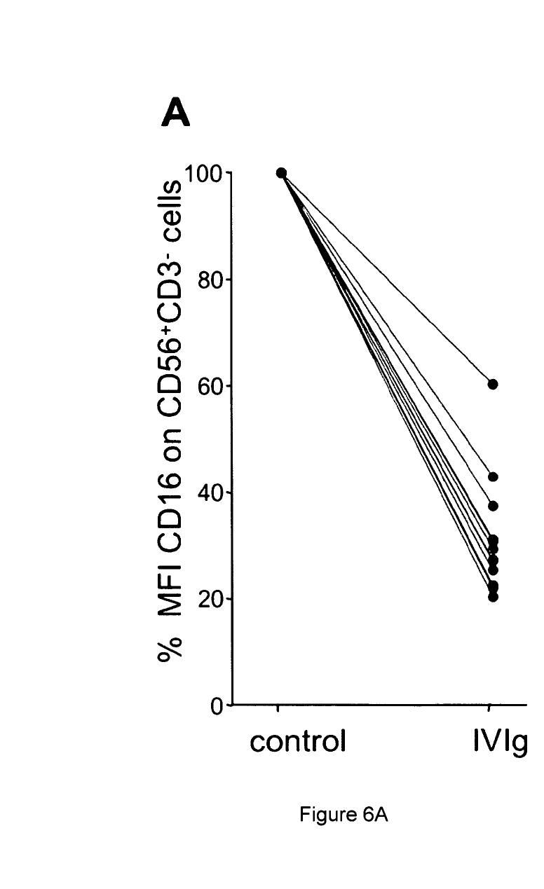

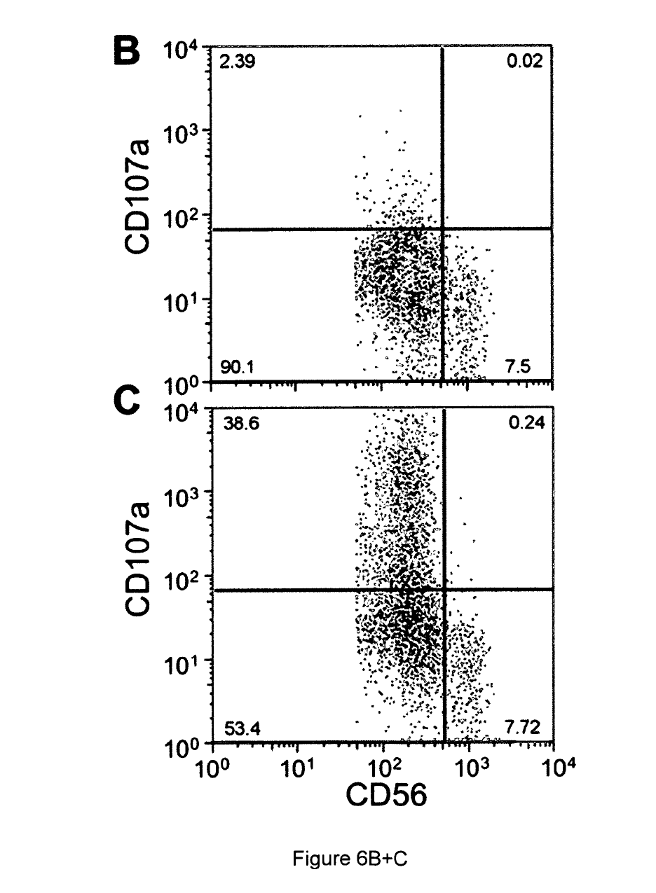

IVIg treatment significantly reduced surface expression of CD16 (p<0.005, n=12, FIG. 6A). These data are consistent with an IVIg-mediated down-modulation of CD16 and suggest a direct engagement of CD16 by IVIg. CD16 is expressed on the CD56^{dim} population of NK cells, which are more cytotoxic compared to their CD56^{bright} counterpart. This suggests that the direct engagement of CD16 by IVIg is responsible for the observed spontaneous degranulation of NK cells. Consistent with this hypothesis, we found that IVIg specifically induced CD107a externalization in the CD56^{dim}/CD16^{positive} NK cell population (FIG. 6B/C).

### Example 2

### Release of Granzyme B, IFN-Gamma and Perforin by IVIG Induced Degranulation

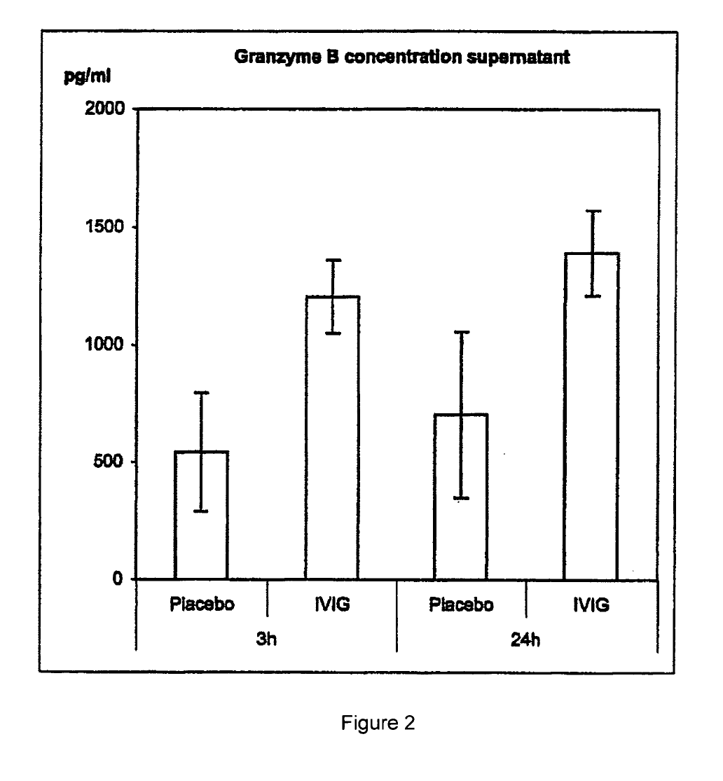

An experimental set-up was chosen to quantify granzyme B in the supernatant of whole blood samples after about 3 h and/or about 24 h (measured by ELISA, Sanquin Reagents, Amsterdam, Netherlands). Release of granzyme B (induced by IgG exposure as described above) led to a marked increase of the granzyme B concentration, as demonstrated in FIG. 2 (3 h, n=6; 24 h n=8).

Measurements of IFN-gamma and perforin were performed by ELISA from the supernatants of whole blood cultures received through centrifugation after about 3 hours and/or about 24 hours of incubation at about 37° C. If necessary, supernatants were kept at about −80° C. until assay. ELISA was performed using a commercially available kit according to the protocol of the manufacturer (Diaclone, Besancon, France). Control measurements were performed under the same conditions except for addition of Immunoglobulin (IgG).

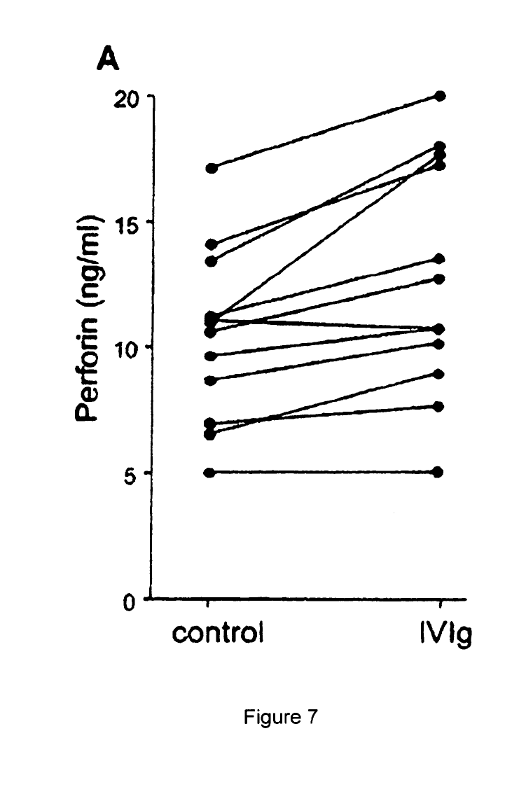

The measurement of granzyme B and perforin in whole blood samples confirmed a spontaneous degranulation after IgG exposure. Consistent with the CD107a data, IVIg induced a significant release of granzyme B (p=0.002; n=12; Wilcoxon Signed-Rank Test was applied for statistical analysis with the SPSS software. P values less than 0.05 were considered significant.) and a more modest increase in perforin (p<0.005; n=12) in the absence of target cells. Results for perforin are demonstrated in FIG. 7.

### Example 3

### Whole Blood Stimulation and Impact of Immunoglobulin Application on IFN-Gamma and IP-10 Levels

Heparinised venous blood from healthy donors (n=19) was collected in 7.5 ml tubes (Sarstedt, Nümbrecht, Germany). A stock solution of 5% IVIG or 10% IVIG (Octagam, Octapharma, Vienna, Austria) was diluted to a concentration of 10 mg/ml IVIG in culture medium (1:5, 1:10 respectively). Placebo solutions according to the immunoglobulin formulations were diluted in the same way as IVIG (1:5 or 1:10 in medium).

Lipopolysaccharide (LPS, Sigma, St. Louis, USA) was diluted to a concentration of 100 ng/ml for stimulation. 1 ml of whole blood was incubated together with 1 ml culture medium or 1 ml culture medium containing placebo or 1 ml culture medium containing 10 mg/ml IVIG in the presence or absence of stimulation by 37° C. for 3 h.

Messenger RNA and cDNA synthesis were performed according to standard methods. An aliquot of 8.2 μl mRNA was reverse transcribed using a first strand cDNA synthesis kit (RAS) and oligo-(dT) as primer using the manufactures protocol in a thermocycler. After termination of the cDNA synthesis, the reaction mix was diluted to a final volume of 500 μl and stored at −20° C. until polymerase chain reaction (PCR) analysis.

Parameter specific primer sets optimized for the LightCycler (RAS) were developed and purchased from SEARCH-LC GmbH (Heidelberg, Germany). The PCR was performed with the LightCycler FastStart DNA Sybr Green I kit (RAS) according to the protocol provided in the parameter specific kits.

RNA input was normalised by the average expression of two housekeeping genes beta-actin and cyclophilin B. The data is presented as adjusted transcripts per μl cDNA.

Cytokine and chemokine release in IVIG stimulated whole blood (versus placebo) was measured after 24 h with a high-sensitivity ELISA (Diaclone, Besancon, France) or Luminex (Multiplex) system (Bio-Plex, Bio-Rad Labaratories, Hercules, Calif., USA).

To measure the release of cytokines and chemokines in LPS-stimulated whole blood after culture for 3 h at 37° C., the samples were centrifuged. The supernatant was immediately collected and stored at −80° C. The supernatants were measured using BD Cytometric Bead Array kits (Human Th1/Th2 cytokine CBA kit, Human Chemokine CBA kit, both BD Biosciences, San Diego, USA), according to the manufactures protocol.

### Results

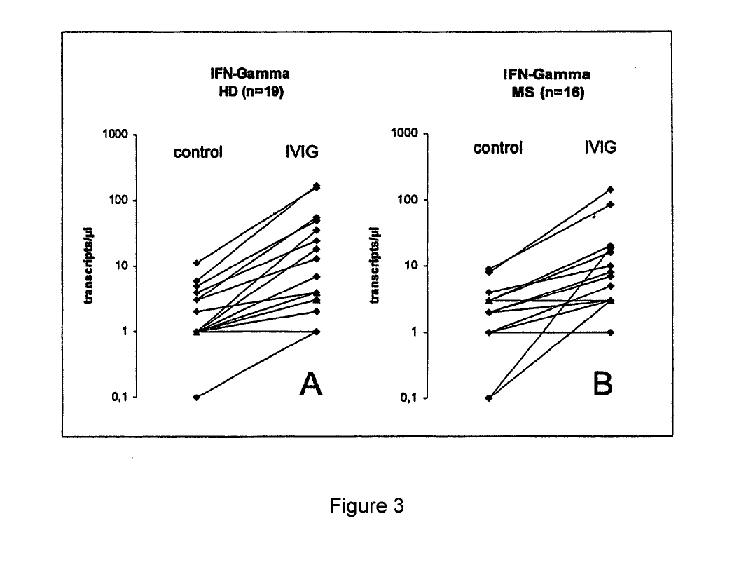

In 18/19 healthy subjects and 14/16 MS patients IFN-gamma gene expression was induced by IVIG, (see FIGS. 3 a) and 3 b).

FIG. 3: Gene expression of IFN-gamma induced by IVIG (transcripts μl) in HD (n=19) and MS patients (n=16).

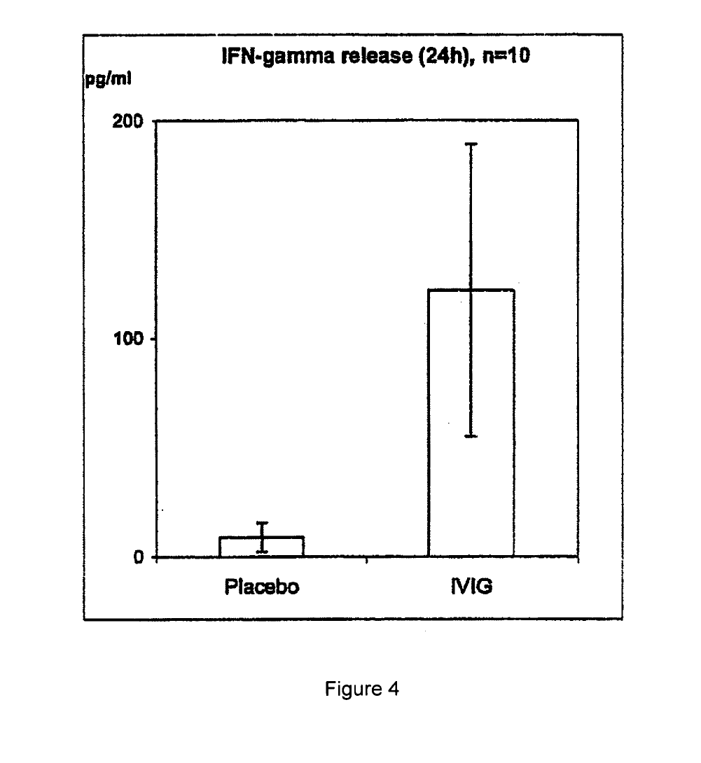

FIG. 4 shows protein release (IFN-gamma) induced by IVIG (high-sensitivity-ELISA) after 24 h, (n=10).

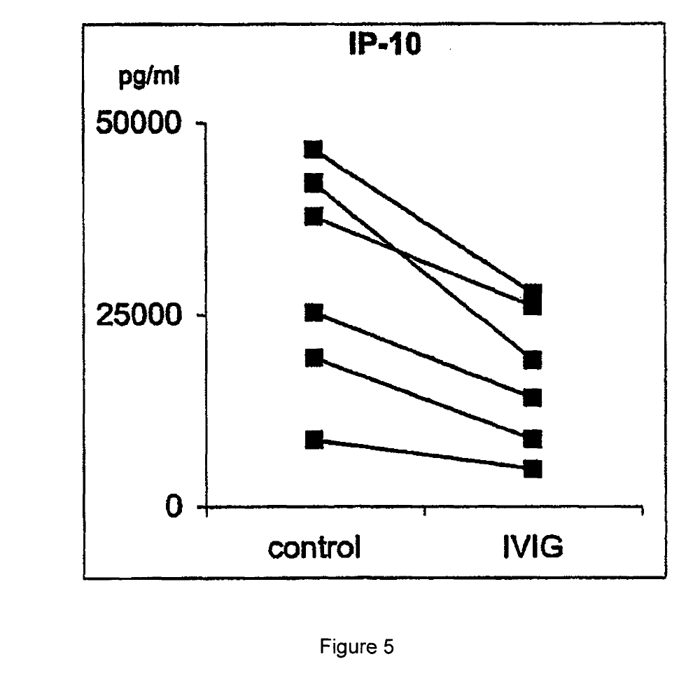

However, IP-10 protein levels (and transcript numbers, not shown) were significantly less increased (n=6) in the presence of IVIG when compared to the control not containing immunoglobulins (settings in the presence of LPS), repealing individual differences in reaction strength to exposure of added immunoglobulin (FIG. 5).

### Example 4

### Assessment of NK Cytotoxicity by ⁵¹Cr Release Assay

Whole blood ⁵¹Cr release assay was performed by dilution of blood in assay medium (IMDM, 10% FCS and 1% penicillin/streptomycin) with IVIg or control for about 3 h at about 37° C. and then distributed on a V-bottom 96-well plate and mixed with ⁵¹Cr labeled K562 target cells at different blood dilutions. Plates were incubated for about 4 h at about 37° C. and supernatant was harvested. ⁵¹Cr release was measured in a gamma counter. To determine the absolute number of NK cells for the calculation of the effector to target ratios, 200 μl diluted blood samples were incubated for about 20 min on ice with PE-conjugated anti-CD56 and PE/Cy-5-conjugated anti-CD3 mAbs. Then 20.000 FITC-conjugated beads (Right Reference Standard, Bangs Laboratories, Fishers, Ind., USA) were added and blood samples were subjected to erythrocyte lysis using FACS Lysing Solution (BD Biosciences, Heidelberg, Germany). NK cell number of diluted blood samples was calculated as [NK cells/μl sample]=[(CD56+/CD3− cell count)/(bead count)]×100.

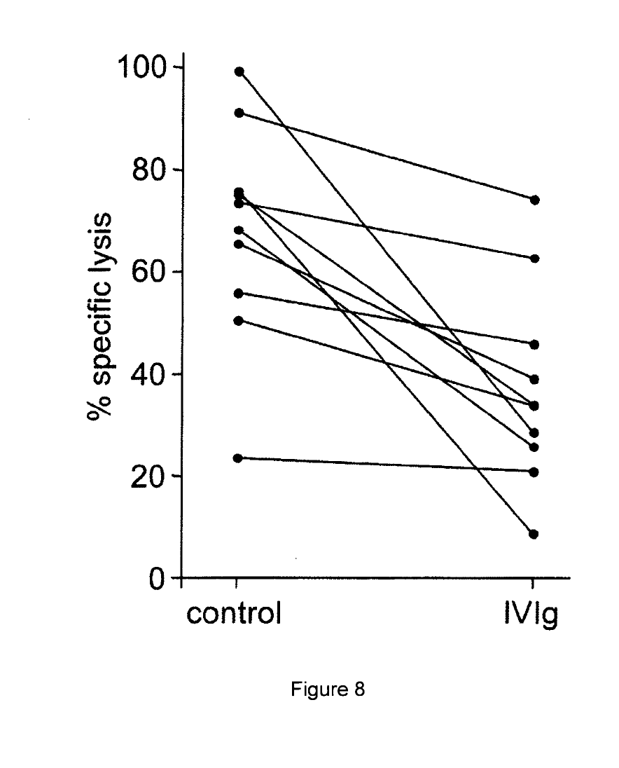

IVIg treatment resulted in a significant reduction of the lysis of the NK target K562 (p<0,005, n=10, FIG. 8).

### LEGENDS TO FIGS. 1, 3-8

FIG. 1: Degranulation of NK cells after whole blood (WB) stimulation by IVIG (n=8).

FIG. 6: Down-modulation of CD16 and degranulation of CD56^{dim} NK cells by IVIg (A) CD16 surface expression (MFI in %, control=100%) on CD56⁺CD3⁻ cells in whole blood after 3 hours of incubation with IVIg or control, n=12 (B, C) CD107a externalization on CD56^{dim} and CD56^{bright} NK cells in whole blood after 3 hours of incubation with IVIg (C) or control (B). Percent of total CD56^{dim} or CD56^{bright} cells expressing CD107a is indicated.

FIG. 7: IVIg induces degranulation of NK cells in whole blood determined as perforin Perforin (A) release (pg/ml) into the supernatant after 24 h incubation of whole blood with IVIg or control (n=12, p<0.005).

FIG. 8: IVIg reduces the killing activity of NK cells in whole blood. Whole blood samples from healthy individuals were treated with IVIg or control for about 3 hours at about 37° C. NK cell activity was then determined by a 4 h ⁵¹Cr release assay against the NK cell sensitive target cell K562. The lysis of K562 cells using 100 μl of whole blood is shown (n=10, p<0.005).

1. A method of determining a patient's susceptibility to immunoglobulins in response to a treatment of a disease or prophylaxis of a disease with NK cell disturbancies with immunoglobulins by determination of NK cell modulation by said immunoglobulins.

2. The method of claim 1 wherein the amount of the degranulation of natural killer cells is determined in an in vitro method.

3. The method of claim 1 wherein the patient having a positive degranulation of natural killer cells is determined as susceptible to a treatment with immunoglobulins.

4. The method of claim 1 wherein determining a patient's susceptibility to immunoglobulins in response to a treatment of a disease or prophylaxis of a disease with immunoglobulins is performed by detection of a transcript level, a protein level or a transcript level and a protein level.

5. The method of claim 4 wherein the transcript level, the protein level or the transcript level and the protein level is quantitatively determined.

6. The method of claim 1 wherein transcript level is determined by nucleic acid detection methods, such as polymerase chain reaction (PCR).

7. The method of claim 5 wherein the protein level is determined using released natural killer cell proteins and/or proteins expressed on the natural killer cell surface.

8. The method of claim 1 wherein whole blood or blood cells are incubated in presence of a stimulant in at least one assay in presence of immunoglobulins and in at least one assay in absence of immunoglobulins as control, followed by measurement of events by expression of factors indicating NK cell modulation selected from the group consisting of IFN-gamma, IP-10, F_{c}-gamma III receptor, granzyme B, perforin, CD107a or combinations thereof for assessing individual responsiveness to the administration of immunoglobulin.

9. The method according to claim 8 wherein the events which are measured are the abundance of transcription and/or protein expression level of the factors, in particular quantitative measurements of the events.

10. The method according to claim 8 wherein the stimulant is selected from the group consisting of lipopolysaccharides (LPS), phorbol-12-myristate-13 acetate (PMA), ionomycin, mAbs binding to cell surface proteins or combinations thereof.

11. The method according to claim 8 wherein the amount of immunoglobulins used in assays is from about 0.01 to about 100 mg/ml, in particular from about 1 to about 50 mg/ml.

12. The method according to claim 1 wherein the disease is selected from the group consisting of inflammatory mediated immune diseases, autoimmune diseases and allergies.
